(19) United States
(12) Patent Application Publication
(10) Pub. No.: US 2025/0346623 A1
(43) Pub. Date: Nov. 13, 2025

(54) VERSATILE SYNTHETIC ROUTE FOR NEUTRAL MORPHOLINO OLIGONUCLEOTIDES WITH PHOSPHORYL GUANIDINIUM (PG) BACKBONE

(71) Applicant: Bio-Synthesis, Inc., Lewisville, TX (US)

(72) Inventors: Kavitha Sudheendran, Lewisville, TX (US); Moussa Barzani, Lewisville, TX (US); Aaron Castro, Lewisville, TX (US); Miguel Castro, Lewisville, TX (US)

(21) Appl. No.: 19/203,725

(22) Filed: May 9, 2025

Related U.S. Application Data
(60) Provisional application No. 63/645,397, filed on May 10, 2024.

(51) Int. Cl. C07H 21/00

(52) U.S. Cl. CPC C07H 21/00

(57) ABSTRACT

Methods featuring phosphoryl guanidinium based backbone and morpholino with phosphoramidite chemistry result in neutral antisense oligonucleotides. The PGMO is composed of a morpholino backbone linked to a phosphoryl guanidinium internucleotide (PG) linkage. These oligonucleotides can be used to treat cancer, autoimmune diseases, and other rare diseases.

### CROSS-REFERENCE TO RELATED APPLICATIONS

This application is a nonprovisional and claims benefit of U.S. Application 63/645,397 filed May 10, 2024, the specification of which is incorporated herein in its entirety by reference.

### FIELD OF THE INVENTION

The present invention features compositions and methods to synthesize uncharged morpholino oligonucleotides having phosphoryl guanidinium linkages.

### BACKGROUND OF THE INVENTION

Nucleic acid therapeutics have been a topic of significant interest in the scientific community for the past 30 years due to their unique ability to regulate gene expression. These drugs are chemically modified oligonucleotides with a sugar backbone, nucleobase, and a bridged phosphate linkage. They can be single-stranded, like antisense oligonucleotide (ASO), or double-stranded, like in siRNA. Based on their mode of action for regulating gene expression, oligonucleotide drugs can be broadly classified as antisense oligonucleotides (ASOs), RNA interference (SIRNA), splice-switching oligonucleotides (SSOs), or RNA aptamers. These drugs can regulate gene expression by blocking mRNA translation, cleaving mRNA by cellular nucleases like RNaseH, switching pre-mRNA splicing, or preventing ribosome recruitment.

Recent developments in oligonucleotide chemistry have significantly improved nucleic acid therapeutics' safety and pharmacokinetics properties. These advancements have increased specificity, binding affinity to the mRNA, improved cellular uptake, and stability of the oligonucleotide drugs for chronic diseases. Consequently, many oligonucleotide drugs have been approved, demonstrating the immense potential of this technology in gene expression modulation. Over the past 20 years, two types of neutral backbone nucleic acid analogs have been studied extensively: phosphorodiamidate morpholinos (PMOs) and peptide nucleic acids (PNAs). Both have exhibited potential activity in binding with natural DNA and RNA. Recent developments have highlighted the significance of various morpholino modifications on different diseases.

As shown in FIG. 1, various morpholino modifications include phosphorodiamidate morpholino (PMO), thiomorpholino (TMO), morpholino oligonucleotide (MO), guanidinium morpholinos (GMO) and phosphoryl guanidinium morpholinos (PGMO). It is interesting to note that among the 24 therapeutic oligonucleotide drugs that have been approved, five of them (Exondys 51® in 2016, Vyondys 53® in 2019, Viltepso® in 2020, Amondys 45® in 2021, Elevidys in 2023) are for the treatment of Duchenne muscular dystrophy and contains morpholino motifs. Several other morpholino oligonucleotide drugs are currently undergoing clinical trials. Previous studies have confirmed that PMO has a high binding affinity to its target mRNA, leads to fast plasma clearance, is sequence-specific, soluble in water, and has low toxicity. However, all morpholino antisense oligonucleotides developed so far are phosphorodiamidate morpholino nucleotides (PMO).

In the field of PMO synthesis, previous works have proposed the reverse synthesis (5′ to 3′ approach) using a chlorophosphoramidate as the precursor. Since then, further developments have been made with the reverse synthesis using P(V) chemistry and H phosphonate approach. It has also been reported that an automated oligo synthesizer used the modified P(V) chemistry to synthesize PMO. The synthesis of PMOs on a large scale is limited due to the low reactivity of monomers and the long reaction times required to complete the condensation reaction. Additionally, the PMO syntheses are not compatible with PMO-DNA/RNA chimeras and have not been reported with the standard solid-phase synthesis via P(III) chemistry.

Modification of internucleotide linkages is of particular interest due to the significant improvement in physicochemical properties, which can modulate plasma stability, binding properties, solubility, cellular uptake, and ultimately biological activity of nucleic acids. Previous works featured a new method for synthesizing a PMO analog called thiomorpholino oligonucleotide (TMOs) with thiophosphate internucleotide linkages on morpholino. This method is compatible with standard automated solid-phase phosphoramidate synthesis conditions via P(III) approach. It allows for the preparation of fully modified thiomorpholinos and hybrid morpholinos that carry DNA/modified RNA oligonucleotides with phosphorothioate linkages. These negatively charged TMOs exhibited high RNA binding affinity and nuclease stability. Chimeric TMOs show potential as novel drug candidates for nucleic acid therapeutics. Although this method is useful for synthesizing TMOs, it cannot synthesize morpholino oligonucleotides with uncharged phosphorus-nitrogen linkages.

In another previous work, a phosphoramidate P(V) chemistry method was developed using 1,8-Diazabicyclo [5.4.0] undec-7-ene (DBU) as an activator/coupling agent to synthesize chiral PGMO chimeras, which is sensitive to other special modifications on the oligo. However, there is no known procedure for preparing morpholinos with neutral phosphorus internucleotide linkages using standard solid phase synthesis via phosphoramidite P(III) chemistry. Hence, the development of morpholino chimeras with appropriate modifications remains a crucial challenge.

The Staudinger reaction is a highly efficient method for obtaining neutral phosphorus-nitrogen modifications of the inter-nucleotide bond. In conventional solid-phase synthesis of oligonucleotides, potential splice-switching agents such as phosphoryl guanidinium and mesyl phosphoramidate groups with neutral phosphorus-nitrogen modifications have been previously introduced using organic azides in the oxidation step via Staudinger reaction. Recent reports suggest that oligonucleotides modified with phosphorylguanidinium (PG) have shown significant improvements in pharmacological efficacy. These modified oligonucleotides have a high affinity for RNA, improves nuclease resistance in serum protein, increased selectivity, and can detect mutant DNA at lower concentrations. They are also more efficient in penetrating the bacterial cell wall.

The application of PG internucleotide linkage extends to allele-specific real-time PCR analysis. A phosphoryl guanidinium modified oligonucleotide, named 2′-O Me PGO, was designed to target the alanine dehydrogenase-encoding ald gene in Mycobacterium smegmatis. This oligonucleotide inhibited the growth of the bacteria and reduced ald expression at both the transcriptional and translational levels. Interestingly, this effect was achieved through an RNase H-independent mechanism. The biological activity of the phosphoryl guanidinium modified oligonucleotide was found to be higher than its phosphorothioate oligonucleotide counterpart. To overcome multidrug resistance in tumor cells, gapmers were used to target MDR1 mRNA, which silenced it efficiently and increased the sensitivity of tumor cells to chemotherapeutics. The new RNA analog, 2′-OMe PGO, exhibits observed antisense activity and efficient uptake by intracellular microorganisms, potentially advancing tuberculosis treatment and preventing drug-resistant mycobacterial strains.

Tetra alkyl guanidinium groups exhibit hydrophobic properties, leading to longer retention time (_{T}R) of oligonucleotides compared to unmodified ones. The structural, thermodynamic, and kinetic properties of G-quadruplexes formed by oligodeoxynucleotides containing phosphoryl guanidinium have been evaluated and found to be compatible with G-quadruplex formation. A gene silencing experiment found that the siRNA remained active if the passenger strand contained PG modifications. However, the PG group created hindrances, which made it difficult for nucleases to access neighboring sites. The silencing effect was negated when the guide strand was modified with PG groups. Prior works have proposed that adding nucleic acids containing the PG group to the passenger strand could eliminate off-target effects caused by the passenger strand unintentionally entering RISC. However, it is currently possible to make only a limited number of modifications using phosphoryl guanidinium (PG). Hence, there is a need for novel methods of synthesizing morpholino oligonucleotides with PG-based backbones.

### BRIEF SUMMARY OF THE INVENTION

It is an objective of the present invention to provide compositions and methods for synthesizing and characterizing morpholino oligonucleotides with phosphoryl guanidinium backbones, which can be used as an antisense oligonucleotide (ASO) or siRNA to treat, prevent, or inhibit the progression of diseases synthesizing, as specified in the independent claims. Embodiments of the invention are given in the dependent claims. Embodiments of the present invention can be freely combined with each other if they are not mutually exclusive.

In some embodiments, the present invention features PG-based backbones and morpholino with phosphoramidite chemistry. In a non-limiting embodiment, the present invention features a unique chimeric antisense oligonucleotide containing 3′-phosphoryl guanidinium morpholino and modified DNA/RNA nucleotides with phosphorothioates (PS) linkage and vice versa. These chimeric ASOs are promising candidates to treat cancer, autoimmune diseases, and other rare diseases.

Although PMOs have a high binding affinity to target mRNA, sequence specificity, solubility in water, and low toxicity, they cannot enter a cell without a transfecting agent. Without wishing to limit the present invention to a particular theory or mechanism, guanidinium groups can enhance cell membrane permeability via interactions with membrane proteins or inducing transient membrane disruptions. Based on the self-cell penetrating competence of GMO-PMO oligonucleotides and the properties of the phosphoryl guanidinium group and morpholino backbone, the present invention incorporated a morpholino oligonucleotide with phosphoryl guanidinium on the oligonucleotide and used conventional solid-phase synthesis to make the synthesis process easier.

Developing a conventional synthetic procedure is important for PMO synthesis from a chemical viewpoint. However, the synthesis of morpholino oligonucleotides is also crucial due to the instability of phosphorus-nitrogen bond of morpholino ring. Without wishing to be bound to a particular theory or mechanism, the inventor hypothesized that replacing the iodine oxidation step for non-bridging PO linkages in morpholino phosphoramidates with phosphoryl guanidinium might improve hydrolytic stability under acidic conditions. This combination offers the benefits of both phosphorodiamidate morpholino (PMO) and PG linkages, making it a promising candidate for various diseases. None of the presently known prior references or works have this unique and inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows non-limiting examples of morpholino-modified oligonucleotides.

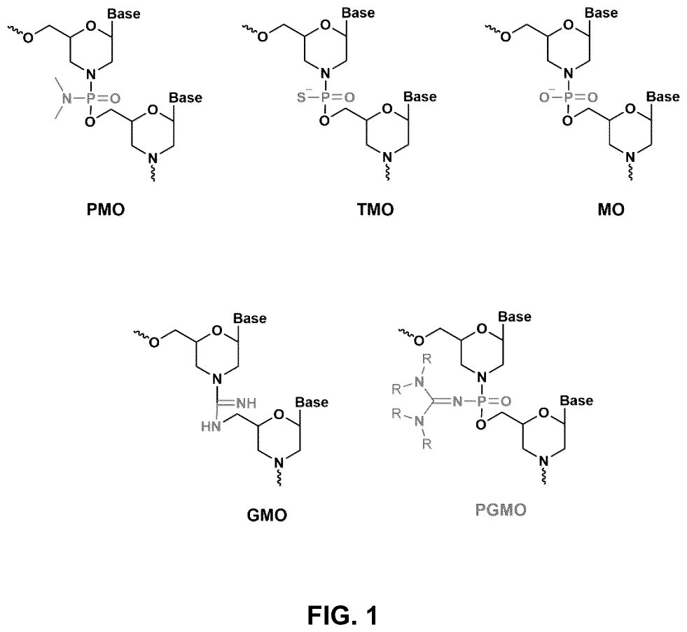

FIG. 2 shows a non-limiting example of a synthetic cycle involved in the phosphoryl guanidinium morpholino synthesis.

FIGS. 3A-3B show TIC and ESI spectra, respectively, for Sequence 2 in Table 2.

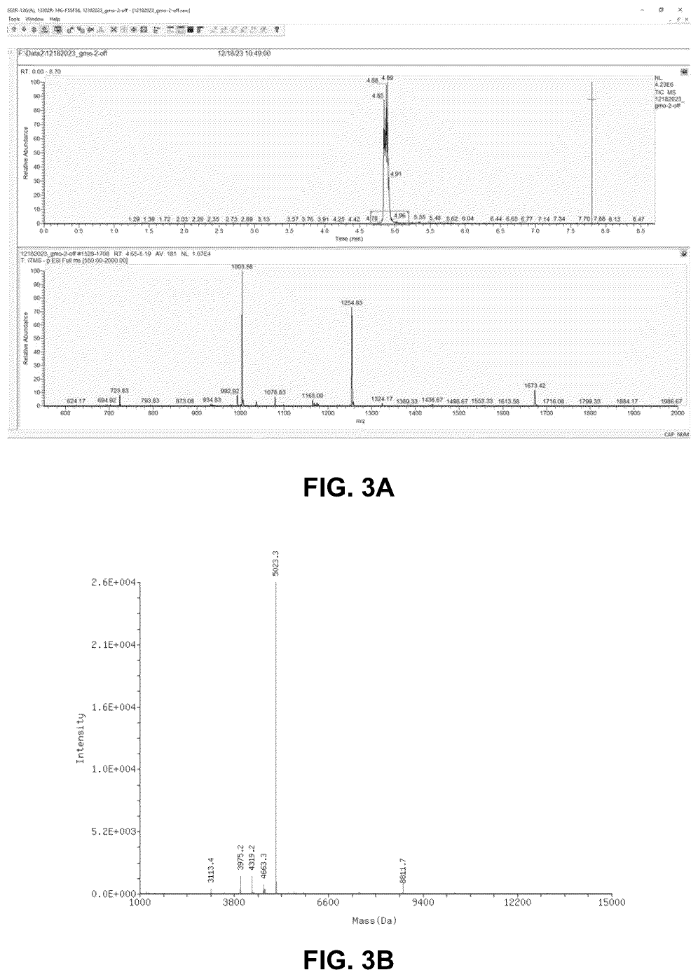

### DETAILED DESCRIPTION OF THE INVENTION

The FDA has approved five drugs that were developed using phosphorodiamidate morpholinos (PMOs). PMOs are RNA analogs with a morpholino backbone that carries no charge. However, the synthesis of PMOs differs from regular phosphoramidite chemistry, which limits their application in synthesizing DNA/RNA chimeras with PMO and their large-scale production. Though the PMOs are site-specific, low cell uptake of PMOs has been reported.

Currently, guanidinium-rich cellular transporters are used for PMO delivery by conjugating PMO with either cell-penetrating peptides (CPPs) or cationic dendrimers to form PPMOs or vivo PMOs. CPPs are peptide-based transporters that peptidases can break down. In order to transfer genetic material into cells, it is necessary to have eight to twelve positively charged guanidinium groups. However, these cationic groups can also interact with negatively charged biomolecules, which can lead to toxicity. Another major hurdle for cellular uptake of a chemically modified oligo is its high negative charge. Therefore, it is important to modify the site-specific nucleotides, which are less toxic and can be easily taken up by cells using regular phosphoramidite chemistry.

A recent study has shown that a DNA and RNA analog of phosphoryl guanidinium oligonucleotide with PG linkage has high RNA affinity and increased cellular uptake. The PGMO contains a PG linkage with morpholino backbone and is a promising option for therapeutic purposes. However, generating chimeras containing morpholino nucleotides with therapeutically appropriate modifications is challenging due to the limited synthetic routes available.

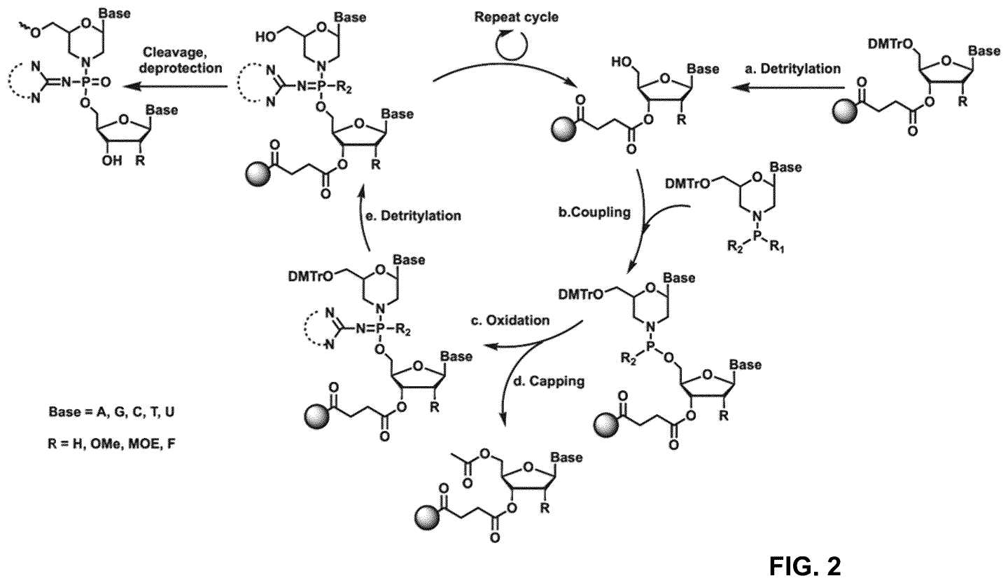

Referring now to FIG. 2, the present invention features a novel approach to produce uncharged morpholino analogs called phosphoryl guanidinium morpholinos (PGMO). The PGMO technique holds immense potential for advancing the antisense oligonucleotide field. The PGMO is composed of a morpholino backbone linked to a phosphoryl guanidinium internucleotide (PG) linkage. This approach is appropriate for synthesizing PGMO-TMO or PGMO-Ps-DNA/Ps-RNA chimeras.

According to some embodiments, the present invention features a method of synthesizing an oligonucleotide with neutral internucleotide linkages. In one embodiment, the method may comprise a) removing a protecting group from a solid supported nucleotide compound comprising a solid support and a first nucleotide; b) coupling the solid supported nucleotide compound with a first phosphordiamidite compound comprising a protecting group, a second nucleotide and a morpholino to produce a dinucleotide intermediate having a P(III) linkage; c) converting the P(III) linkage of the dinucleotide intermediate to a P(V) linkage; d) capping the solid support; e) removing the protecting group from the dinucleotide intermediate; f) repeating steps b.-e. until an oligonucleotide intermediate is formed comprising a desired number of nucleotides; g) cleaving the solid support from the oligonucleotide intermediate; and h) deprotecting the oligonucleotide intermediate to produce the oligonucleotide with neutral internucleotide linkages.

In some embodiments, the steps of removing the protecting group comprises detritylation. The protecting groups may be DMTr. In other embodiments, the coupling step comprises a condensation reaction. In some other embodiments, the converting step comprises either oxidizing or sulfurizing the dinucleotide intermediate.

In some embodiments, the solid supported nucleotide comprises CPG-500 support. In other embodiments, the desired number of nucleotides ranges from about 5 to about 35. In some embodiments, the nucleotides comprise a base. The base may be A, G, C, T, U, or other modified nucleobases. In other embodiments, the P(V) linkages are phosphorothioate, phosphodiester, or phosphoryl guanidinium.

According to other embodiments, the present invention features a method of synthesizing an oligonucleotide with neutral internucleotide linkages. The method may comprise: a) coupling a solid supported nucleotide compound with a first phosphordiamidite compound comprising a protecting group, a second nucleotide and a morpholino to produce a nucleotide intermediate having a P(III) linkage; and b) converting the P(III) linkage of the nucleotide intermediate to a P(V) linkage. In some embodiments, the converting step comprises either oxidizing or sulfurizing the nucleotide intermediate. In some other embodiments, the method may further comprise cleaving the solid support from the oligonucleotide intermediate, and deprotecting the oligonucleotide intermediate to produce the oligonucleotide with neutral internucleotide linkages.

According to some embodiments, the present invention features an uncharged oligonucleotide comprising a plurality of morpholino nucleotides and phosphoryl (V) linkages, where each linkage is bridging two morpholino nucleotides. In some embodiments, the number of nucleotides ranges from about 5 to about 35. In other embodiments, the nucleotides comprise a base. The base may be A, G, C, T, U, or other modified nucleobases. In some embodiments, the P(V) linkages are phosphorothioate, phosphodiester, or phosphoryl guanidinium.

In some embodiments, the uncharged oligonucleotide is a product of a solid phase phosphoramidite P(III) synthesis. In a non-limiting embodiment, the solid phase phosphoramidite P(III) synthesis may comprise: a) removing a protecting group from a solid supported nucleotide compound comprising a solid support and a first nucleotide; b) coupling the solid supported nucleotide compound with a first phosphordiamidite compound comprising a protecting group, a second nucleotide and a morpholino to produce a dinucleotide intermediate having a P(III) linkage; c) converting the P(III) linkage of the dinucleotide intermediate to a P(V) linkage; d) capping the solid support; e) removing the protecting group from the dinucleotide intermediate; f) repeating steps b.-e. until an oligonucleotide intermediate is formed comprising a desired number of nucleotides; g) cleaving the solid support from the oligonucleotide intermediate; and h) deprotecting the oligonucleotide intermediate to produce the uncharged oligonucleotide.

According to some embodiments, the present invention features an uncharged, solid supported oligonucleotide comprising a terminal nucleotide coupled to a solid support, at least one morpholino nucleotide, and at least one phosphoryl (V) linkage bridging two nucleotides. In some embodiments, the number of nucleotides ranges from about 5 to about 35. In other embodiments, the nucleotides comprise a base. The base may be A, G, C, T, U, or other modified nucleobases. In some embodiments, the P(V) linkage is phosphorothioate, phosphodiester, or phosphoryl guanidinium.

In some embodiments, the uncharged, solid supported oligonucleotide is produced using solid phase phosphoramidite P(III) synthesis. In some embodiments, the uncharged, solid supported oligonucleotide is an intermediate of the solid phase phosphoramidite P(III) synthesis. In a non-limiting embodiment, the solid phase phosphoramidite P(III) synthesis may comprise: a) removing a protecting group from a solid supported nucleotide compound comprising the terminal nucleotide coupled to the solid support; b) coupling the solid supported nucleotide compound with a first phosphordiamidite compound comprising a protecting group, a second nucleotide and a morpholino to produce a dinucleotide intermediate having a P(III) linkage; c) converting the P(III) linkage of the dinucleotide intermediate to a P(V) linkage; d) capping the solid support; e) removing the protecting group from the dinucleotide intermediate; and f) repeating steps b.-e. until the uncharged oligonucleotide comprises a desired number of nucleotides.

### EXAMPLE

The following is a non-limiting example of the present invention. It is to be understood that said example is not intended to limit the present invention in any way. Equivalents or substitutes are within the scope of the present invention.

### Solid Phase Synthesis, HPLC Purification and LC/MS Analysis of PGMO-TMO/DNA/RNA Chimeras

First, the 5′-DMT protecting group was removed from the solid supported 2′-methoxyribonucleoside (CPG-500 support, Glen Research) using 3% dichloroacetic acid in dichloromethane. Condensations were performed between CPG-linked 5′-hydroxyl-2′-methoxyribonucleoside of mABz, mGiBu, mCBz, mT or 5′-hydroxyl-2′-deoxyribonucleoside and 6′-DMT-morpholino nucleoside in anhydrous acetonitrile containing 0.2M 5-ethylthio-1H-tetrazole (ETT) as an activator for 100 seconds.

Phosphoroguanidinium morpholino amidate (PGMO) or thiomorpholino amidate (TMO) or 2′-methoxyphosphorothioate or 2′-deoxyphosphorothioate were obtained from the conversion of P(III) linkages to P(V) in 60 seconds using oxidation and sulfurization agents. The hydroxyl groups that were not reacted were acetylated using conventional capping reagents. Cap A was tetrahydrofuran/acetic anhydride, and Cap B was 16% 1-Methylimidazole in pyridine/tetrahydrofuran as shown in Table 1. The DMT protecting group on the resulting dinucleotide was removed using the deblocking mixture, and this cycle was repeated numerous times until the final synthesis of the PGMO/TMO or PGMO/TMO-DNA-RNA chimera was complete. The DMT-ON oligo was removed from the synthesizer and used for subsequent processing.

TABLE 1
Chemical steps used in the automated synthesizer for
the preparation of PGMO- TMO/DNA/RNA chimeras.

| | | Time | |
|---|---|---|---|
| Reactions | Reagents/solvents | (seconds) | |
| Detritylation | 3% dichloroacetic acid in dichloromethane | 210 | Sec |
| Condensation | 0.07M phosphoramidite + activator | 100 | sec |
| | (0.20M ETT in anhydrous acetonitrile) | | |
| Oxidation | Guanidinium reagent | 60 | sec |
| Sulfurization | Sulfurizing reagent | 60 | sec |
| Capping | Cap A: Tetrahydrofuran/Acetic anhydride | 10 | sec |

Cap B: 16% 1-Methylimidazole in
pyridine/Tetrahydrofuran

The 5′-DMT ON oligonucleotides were cleaved from the solid support and deprotected in AMA for 3 hours at room temperature. After the cleavage, the CPG was filtered out and the solution was evaporated to dryness using a SpeedVac (Thermo Fisher Scientific). The crude sample was analyzed by ESI, and the remaining solution was purified using RP-HPLC column chromatography with a gradient of 10%-50% of acetonitrile (buffer B) and TEAA buffer (buffer A). Pure compounds were combined and then evaporated to dryness. The resulting pure DMT ON samples were analyzed using ESI.

The pure DMT-ON sample was dissolved in 0.5 ml of detritylation mixture. After the detritylation process, the mixture was neutralized with 5 μl of triethylamine. Then, the sample was desalted using CentriPure N100 and the final DMT-OFF product was evaporated to dryness on a SpeedVac. The purity of the product was determined by ESI analysis and analytical RP-HPLC, while the concentration of the sample was measured by nanodrop before storing the samples at −20° C.

The following sequences were synthesized successfully, as shown in Table 2.

TABLE 2
List of oligonucleotides and their calculated molecular weight and
experimental molecular weight on ESI. The ESI data for sequence 2 are shown in FIG.
3B. Underlined mrNpn is PGMOs, italicized mrNs is TMOs, dNs is 2′ dna thiophosphate,
and mN is 2′ OMe RNA thiophosphate.

| | | molecular weight | |
|---|---|---|---|
| | | | experimental |
| No. | 5′-3′ oligonucleotide sequence | Calc. m/z | m/z |
| 1 | mrGpn mrTpn mrCpn dCs dAs dGs dCs dCs dCs | 5260.24 | 5261.2 |
| | dCs dAs mrTpn mrG pn mrGpn mA | | |
| 2 | mrGpn mrTpn mrCpn dCs dAs dGs dCs dCs dCs | 5023.01 | 5023.3 |
| | dCs dAs mrTs mrGs mrGs mA | | |
| 3 | mrGs mrTpn mrCpn dCs dAs dGs dCs dCs dCs | 5022.94 | 5023.7 |
| | dCs dAs mUs mGs mGs mA | | |

Purification of oligonucleotides by RP-HPLC: For semi-preparative purification of crude DMT-ON oligonucleotides, ion-pair reverse-phase HPLC technique was used by employing a Schimadzu HPLC system. The stationary phase consisted of a Phenomenex Clarity oligo RP with a 10 μm particle size and 10 mm i.d.×250 mm dimension. The elution of the oligonucleotide was observed through the absorption at 254, 280, and 300 nm wavelengths. Buffer A (50 mM TEAA in water) and Buffer B (100% acetonitrile) were used as the mobile phase to generate a gradient of 0-100% of Buffer B over 45 minutes at a flow rate of 6 mL/min. Purity of the sample was measured using ESI and the combined pure fractions were evaporated to dryness using a SpeedVac Vacuum Concentrator (Thermo Fisher Scientific). DMT-Off PGMO oligonucleotide chimeras that were obtained by the treatment of pure DMT-ON ODNs with detritylation mixture and were desalted to obtain pure oligonucleotides with greater than 90% purity.

ESI analysis: ESI analysis was performed using a Thermo LTQ XL linear ion trap. The aqueous phase comprised Buffer A which contained 976 ml water, 20 ml hexafluoro-2-propanol (HFIP), and 4 ml triethylamine. On the other hand, the organic phase was made up of Buffer B which had 976 ml methanol, 20 ml hexafluoro-2-propanol (HF IP), and 4 ml triethylamine. The observed masses of the oligonucleotides were consistent with their expected theoretical masses.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of synthesizing an oligonucleotide with neutral internucleotide linkages, said method comprising:
a. removing a protecting group from a solid supported nucleotide compound comprising a solid support and a first nucleotide;
b. coupling the solid supported nucleotide compound with a first phosphordiamidite compound comprising a protecting group, a second nucleotide and a morpholino to produce a dinucleotide intermediate having a P(III) linkage;
c. converting the P(III) linkage of the dinucleotide intermediate to a P(V) linkage;
d. capping the solid support;
e. removing the protecting group from the dinucleotide intermediate;
f. repeating steps b.-e. until an oligonucleotide intermediate is formed comprising a desired number of nucleotides;
g. cleaving the solid support from the oligonucleotide intermediate; and
h. deprotecting the oligonucleotide intermediate to produce the oligonucleotide with neutral internucleotide linkages.

2. The method of claim 1, wherein the steps of removing the protecting group comprises detritylation, wherein the protecting groups are DMTr.

3. The method of claim 1, wherein the coupling step comprises a condensation reaction.

4. The method of claim 1, wherein the solid supported nucleotide comprises CPG-500 support.

5. The method of claim 1, wherein the desired number of nucleotides ranges from about 5 to about 35.

6. The method of claim 1, wherein the nucleotides comprise a base, wherein the base is A, G, C, T, U, or other modified nucleobases.

7. The method of claim 1, wherein the converting step comprises either oxidizing or sulfurizing the dinucleotide intermediate.

8. The method of claim 1, wherein the P(V) linkages is phosphorothioate, phosphodiester, or phosphoryl guanidinium.

9. A method of synthesizing an oligonucleotide with neutral internucleotide linkages, said method comprising:
a. coupling a solid supported nucleotide compound with a first phosphordiamidite compound comprising a protecting group, a second nucleotide and a morpholino to produce a nucleotide intermediate having a P(III) linkage; and
b. converting the P(III) linkage of the nucleotide intermediate to a P(V) linkage.

10. The method of claim 9, wherein the converting step comprises either oxidizing or sulfurizing the nucleotide intermediate.

11. The method of claim 9, further comprising:
a. cleaving the solid support from the oligonucleotide intermediate; and
b. deprotecting the oligonucleotide intermediate to produce the oligonucleotide with neutral internucleotide linkages.

12. An uncharged oligonucleotide comprising a plurality of morpholino nucleotides and phosphoryl (V) linkages, each linkage bridging two morpholino nucleotides, wherein said uncharged oligonucleotide is a product of a solid phase phosphoramidite P(III) synthesis.

13. The oligonucleotide of claim 12, wherein the number of nucleotides ranges from about 5 to about 35.

14. The oligonucleotide of claim 12, wherein the nucleotides comprise a base, wherein the base is A, G, C, T, U, or other modified nucleobases.

15. The oligonucleotide of claim 12, wherein the P(V) linkages is phosphorothioate, phosphodiester, or phosphoryl guanidinium.

16. The oligonucleotide of claim 12, wherein the solid phase phosphoramidite P(III) synthesis comprises:
a. removing a protecting group from a solid supported nucleotide compound comprising a solid support and a first nucleotide;
b. coupling the solid supported nucleotide compound with a first phosphordiamidite compound comprising a protecting group, a second nucleotide and a morpholino to produce a dinucleotide intermediate having a P(III) linkage;
c. converting the P(III) linkage of the dinucleotide intermediate to a P(V) linkage;
d. capping the solid support;
e. removing the protecting group from the dinucleotide intermediate;
f. repeating steps b.-e. until an oligonucleotide intermediate is formed comprising a desired number of nucleotides;
g. cleaving the solid support from the oligonucleotide intermediate; and
h. deprotecting the oligonucleotide intermediate to produce the uncharged oligonucleotide.

17. An uncharged, solid supported oligonucleotide comprising a terminal nucleotide coupled to a solid support, at least one morpholino nucleotide, and at least one phosphoryl (V) linkage, wherein the P(V) linkage bridges two nucleotides, wherein said uncharged oligonucleotide is produced using solid phase phosphoramidite P(III) synthesis.

18. The oligonucleotide of claim 17, wherein the number of nucleotides ranges from about 5 to about 35.

19. The oligonucleotide of claim 17, wherein the nucleotides comprise a base, wherein the base is A, G, C, T, U, or other modified nucleobases.

20. The oligonucleotide of claim 17, wherein the P(V) linkage is phosphorothioate, phosphodiester, or phosphoryl guanidinium.

21. The oligonucleotide of claim 17, wherein the solid phase phosphoramidite P(III) synthesis comprises:
a. removing a protecting group from a solid supported nucleotide compound comprising the terminal nucleotide coupled to the solid support;
b. coupling the solid supported nucleotide compound with a first phosphordiamidite compound comprising a protecting group, a second nucleotide and a morpholino to produce a dinucleotide intermediate having a P(III) linkage;
c. converting the P(III) linkage of the dinucleotide intermediate to a P(V) linkage;
d. capping the solid support;
e. removing the protecting group from the dinucleotide intermediate; and
f. repeating steps b.-e. until the uncharged oligonucleotide comprises a desired number of nucleotides.
